(19) United States
(12) Patent Application Publication
(10) Pub. No.: US 2026/0144933 A1
(43) Pub. Date: May 28, 2026

(54) METHOD FOR MODIFICATION OF INSULIN DELIVERY DURING PREGNANCY IN AUTOMATIC INSULIN DELIVERY SYSTEMS

(71) Applicant: INSULET CORPORATION, Acton, MA (US)

(72) Inventor: Rangarajan NARAYANASWAMI, Weston, MA (US)

(21) Appl. No.: 19/415,356

(22) Filed: Dec. 10, 2025

Related U.S. Application Data
(63) Continuation of application No. 17/812,066, filed on Jul. 12, 2022, now Pat. No. 12,521,486.
(60) Provisional application No. 63/222,496, filed on Jul. 16, 2021.

(51) Int. Cl. A61M 5/172; G16H 20/17

(52) U.S. Cl. CPC A61M 5/1723; G16H 20/17

(57) ABSTRACT

The disclosed embodiments are directed to methods for dynamically adjusting the total daily insulin requirements of a user during pregnancy, based on the gestational week. An initial estimate of the adjusted total daily insulin requirement may be calculated as a multiple of the pre-pregnancy total daily insulin requirement, based on an average scale factor from a population of pregnant women suffering from Type I diabetes mellitus. An automatic drug delivery device may adjust the initial estimate of the total daily insulin requirement based on blood glucose level readings from a continuous glucose monitor during the course of the pregnancy.

### RELATED APPLICATIONS

This application is a continuation of U.S. patent application Ser. No. 17/812,066, filed Jul. 12, 2022, which claims the benefit of U.S. Provisional Ser. No. 63/222,496, filed Jul. 16, 2021, the contents of which are incorporated herein by reference in their entirety.

### BACKGROUND

Conventional automatic drug delivery systems may deliver a medication, for example, insulin, over the course of a day via one or more basal dosages and/or one or more bolus dosages. Control of the automatic drug delivery system to deliver the required basal and/or bolus dosages of the medication may be accomplished in accordance with a dosing algorithm which configures the size and timing of both basal and bolus dosages of the medication.

For people with Type 1 diabetes mellitus, all or a portion of their daily insulin requirements may be provided by the automatic drug delivery system. The automatic drug delivery system may be provided with a total daily insulin (TDI) requirement for the user and may calculate basal and bolus doses based on the TDI. TDI represents the aggregate amount of insulin needed by the user for a day. TDI is typically calculated based on the weight of the user. A commonly used rule-of-thumb is that TDI in units of insulin equals user weight in pounds divided by 4. Thus, the TDI for a 120-pound woman is 120 divided by 4 or approximately 30 units of insulin.

Basal dosages of insulin are delivered on an-ongoing basis and address a substantial portion of the need for the insulin on an on-going basis. The dosing algorithm of the automatic drug delivery system may calculate a basal dosage based on the user's TDI. The basal dosage is conventionally determined to be one half of the TDI.

The bolus doses of insulin are delivered when requested by a user or when the dosing algorithm for the automatic drug delivery system concludes, based on information regarding the user, that there is a need to deliver the bolus dosage. For example, the dosing algorithm may receive blood glucose readings indicative of the ingestion of a meal by the user. The magnitude of the post-prandial increase in blood glucose concentration is related to the quantity of carbohydrates ingested during the meal. Thus, a dosing algorithm for a conventional automatic drug delivery device may determine what dosage of insulin will compensate for the quantity of carbohydrates ingested. To determine the amount of insulin needed and, hence, the bolus dosage, the dosing algorithm may multiply the quantity of carbohydrates ingested by the insulin-to-carbohydrate ratio (ICR) for the user. The insulin-to-carbohydrate ratio may for example be started at 1:15, such that 1 unit of insulin is delivered for every 15 grams of carbohydrates ingested and then fine tuned for a user The dosing algorithm may also take into account the current blood glucose concentration, which may be provided by a connected continuous glucose monitor, and the insulin on board (IOB) for the user, which may be calculated based on previous deliveries of insulin and which represents the quantity of insulin delivered to user that still has insulin action remaining.

During pregnancy, glycemic control is vital in optimizing positive pregnancy outcomes for Type I diabetics. Risks of poor glycemic control in pregnancy include congenital malformations, miscarriage, perinatal mortality, maternal preeclampsia, neonatal hypoglycemia, respiratory distress, obstetric interventions at delivery, and macrosomia (larger than average fetal/baby weight).

However, maintenance of good glycemic control is difficult during pregnancy because the insulin requirements are constantly changing, depending on the current phase of the pregnancy. For example, as shown by the graph in FIG. 2, for the average pregnancy experienced by a woman suffering from Type I diabetes, there is an increased risk of hypoglycemia in the first trimester, and significant insulin resistance and, thus, an increased insulin need later in gestation. The total daily dose of insulin per kilogram weight may increase as much as 70% from the first trimester to the third trimester. The insulin to carbohydrate ratio also may decrease throughout the course of the pregnancy. Users on multiple daily injections may reduce their basal insulin in weeks 8 through 12-16 of pregnancy, which is consistent with elevated hypoglycemia risk in the first trimester. The first trimester fall in insulin requirements is thought to be due to a transient reduction in progesterone levels as hormonal production shifts from the corpus luteum to the placenta, as well as decreased prandial requirements due to hyperemesis (morning sickness/nausea/vomiting).

Based on the above, it would be desirable to modify the dosing algorithm for an automatic drug delivery system such as to take into account the changing needs for insulin experienced by women during pregnancy and to deliver modified basal and bolus doses of insulin in accordance with the changing needs in different phases of pregnancy to achieve satisfactory glycemic control.

### DEFINITIONS

As used herein, the term “insulin” should be interpreted to include insulin or co-formulations of two or more of GLP-1, pramlintide, and insulin.

As used herein, the terms “user”, “person” and “patient” are used interchangeably and are meant to include pregnant women suffering from type 1 diabetes mellitus who are using the disclosed invention to control their dosing of insulin during pregnancy.

### SUMMARY

This Summary is provided to introduce a selection of concepts in a simplified form that are further described below in the Detailed Description section. This Summary is not intended to identify key or essential features of the claimed subject matter, nor is it intended as an aid in determining the scope of the claimed subject matter.

Disclosed herein are methods for adjusting the dosing algorithm of an automatic drug delivery system to address the user's changing needs for insulin during various phases of a pregnancy. In a primary embodiment of the invention, this may be accomplished by scaling the pre-pregnancy TDI requirements for a particular user. The scaling factor may vary at various times during course of the pregnancy, for example, on a week to week basis. The insulin may then be administered on a daily basis based on the scaled TDI.

In one aspect of the invention, the automatic detection of the ingestion of a meal by the user may be accomplished by an analysis of the blood glucose curve. The blood glucose curve may be constructed and continuously updated based on readings from a continuous blood glucose monitor auxiliary to or as part of the automatic drug delivery system.

In a second aspect of the invention, a safe bolus dose of insulin may be calculated based on the automatic detection of the ingestion of a meal. The bolus dose of insulin is calculated as a dose that will avoid hypoglycemia in the user.

In a third aspect of the invention, the scaled TDI requirement of the user may be periodically adapted, for example, on a week-by-week basis, for a subsequent week based on a departure from a reference schedule during a current week and the insulin administered in accordance with the adapted TDI requirement. In this aspect of the invention, the basal setting (i.e., the portion of the TDI administered as one or more basal doses) can be set as a percentage of the adapted TDI requirement.

In fourth aspect of the invention, feedback is provided to the user based on the TDI requirements being higher than a reference schedule or lower than a reference schedule, and suggestions can be made for the user regarding the meals, the macronutrient profile of the meals and the amount of exercise performed by the user.

Preferred embodiments of the invention may comprise any combination of the various embodiments or aspects of the invention discussed above.

### BRIEF DESCRIPTION OF THE DRAWINGS

To easily identify the discussion of any particular element, the most significant digit or digits in a reference number refer to the figure number in which that element is first introduced.

FIG. 1 is a block diagram of an exemplary automatic drug delivery system of the type on which the present invention may be implemented.

FIG. 2 is a graph showing, for a typical population of women during the course of a pregnancy suffering from Type I diabetes, (a) the week-by-week average capillary blood glucose readings; (b) a week-by-week average total insulin requirement; and (c) week-by-week average insulin requirement.

FIG. 3 is a table showing a scaling factor for various time intervals during pregnancy to be used to scale a user's pre-pregnancy TDI requirement for various intervals during the pendency of the pregnancy.

FIG. 4 is a schematic diagram graphically showing the analysis of a blood glucose curve to detect factors involved in the automatic detection of the ingestion of a meal by the user.

FIG. 5 is a diagram showing an exemplary embodiment of a decision tree which may be used to determine the ingestion of a meal, taking into account several factors extracted from the blood glucose curve.

FIG. 6 is a flowchart showing the process for the periodic calculation of the meal detection algorithm.

FIG. 7 is a graph showing a histogram showing the quantity of insulin administered for each automatic bolus as a percentage of the TDI.

FIG. 8 is a flowchart showing the calculation of a new basal setting, which is the percentage of the adapted TDI to be administered as basal doses.

FIG. 9 is an exemplary screen provided as an application running as part of the automatic drug delivery system which allows the invocation of pregnancy mode and the specification of the current week of pregnancy and the expected due date.

### DETAILED DESCRIPTION

Methods in accordance with the present disclosure will now be described more fully with reference to the accompanying drawings, where one or more embodiments or various aspects of the invention are shown. The methods may be embodied in many different forms and are not to be construed as being limited to the embodiments set forth herein. Instead, these embodiments are provided so the disclosure will be thorough and complete, and will fully convey the scope of the systems and methods to those skilled in the art. Each of the methods disclosed herein provides one or more advantages over conventional systems and methods.

Persons suffering from Type 1 diabetes may have all or a portion of their daily insulin requirements delivered via an automatic drug delivery system. In some embodiments, the automatic drug delivery system may provide the user's basal dosing requirements, while the user's bolus dosing requirements are delivered manually by the person. In other embodiments, the automatic drug delivery system may provide both basal and bolus doses of insulin.

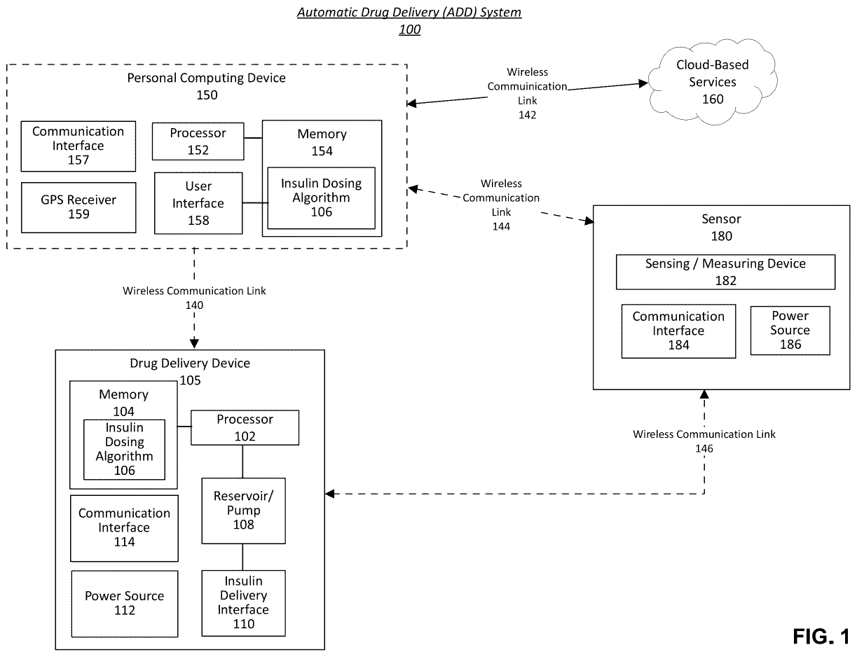

FIG. 1 is a block diagram showing an automatic drug delivery (ADD) system 100 of the type in which various embodiments of the present invention may be implemented. The ADD system 100 includes a insulin dosing algorithm 106 for calculating the basal and bolus dosing requirements of the person and for controlling the mechanical aspects of a drug delivery device 105 responsible for the actual delivery of the insulin to the user. The insulin dosing algorithm 106 may implement the various novel embodiments of the invention described in the Summary above and in more detail below.

In a primary embodiment of the invention, the insulin dosing algorithm 106 may be implemented as software executing on a processor in a drug delivery device 105 as part of an ADD system 100. Drug delivery device 105 may be, for example, a wearable drug delivery device which is discreetly disposed on the body of the user and held in place on the user's skin by an adhesive. Alternatively, drug delivery device 105 may be, for example, a drug delivery device carried by a user (e.g., on a belt or in a pocket) and having an infusion set with tubing connecting the drug delivery device 105 to a cannula that penetrates the user's skin.

Drug delivery device 105 may include a processor 102 in communication with memory 104 containing insulin dosing algorithm 106 which is executed by processor 102. In accordance with insulin dosing algorithm 106, processor 102 may control one or more reservoirs/pumps 108 suitable for delivering insulin to the user via insulin delivery interface 110 which may be, for example, a subcutaneous cannula extending from one or more housings of drug delivery device 105 and into the body of the user. Drug delivery device 105 may further include a wireless communication interface 104. The overall drug delivery device 105 may be powered by power source 112, which may be, for example, batteries or a power harvesting apparatus.

ADD system 100 may further include a sensor 180 which may comprise a sensing/measuring device 182 which may be, for example, a continuous glucose monitor (CGM). Like drug delivery device 105, sensor 180 may also be a wearable device disposed on the body of the user. Sensor 180 may include power source 186 and a wireless communication interface 184.

Drug delivery device 105 and sensor 180 may communicate with each other via wireless communication link 146. Sensor 180 may provide periodic readings of the blood glucose level of the user to drug delivery device 105 for use by the insulin dosing algorithm 106 via wireless communication link 146. For example, sensor 180, in one embodiment, may provide blood glucose level readings to drug delivery device 105 every 5 minutes. Other intervals for reporting the blood glucose levels of the user are within the scope of the invention. In some embodiments of the present invention, the periodic readings of the blood glucose level of the user may be used, as described below, to modify the TDI requirement and the basal/bolus split during various phases of pregnancy.

In alternate embodiments, insulin dosing algorithm 106 may execute on a personal computing device 150 instead of on drug delivery device 105 and may communicate the basal and bolus dosing requirements to drug delivery device 105 via wireless communication link 140. Personal computing device 150 may be configured with a processor 152 and a memory 154 containing software embodying the insulin dosing algorithm 106 for execution on processor 152. Personal computing device 150 may be further configured with a user interface 158 which may be used by the insulin dosing algorithm 106 to enable interaction with a user.

In some embodiments, personal computing device 150 may comprise, for example, a smartphone, a tablet device, a smartwatch, a dedicated personal diabetes manager, or any other personal mobile computing device capable of executing insulin dosing algorithm 106 and communicating with drug delivery device 105, cloud-based services 160 and sensor 180 via any well-known wireless communication protocol.

Insulin dosing algorithm 106 may have a user interface 158 which may be used, for example, to initiate pregnancy mode in insulin dosing algorithm 106, to allow the user to input information necessary to ascertain the current phase of the pregnancy, and, if not already known to insulin dosing algorithm 106, allowing the user to enter their pre-pregnancy TDI requirement. Additionally, user interface 158 may be used to provide feedback to the user regarding the delivery of the insulin, the sensed blood glucose level readings and the effects of exercise and meals ingested by the user on the user's blood glucose levels. In some embodiments, the blood glucose level readings may be received from sensor 180, directly via communication link 144 or indirectly via drug delivery device 105 via wireless communication link 140.

In alternative embodiments wherein no sensor 180 is used, the user may manually take blood glucose readings and enter the blood glucose readings directly via user interface 158. The blood glucose readings may thereafter be transmitted to drug delivery device 105 via wireless communication link 140 for use by basal dosing application 106.

In some embodiments, the functionality of insulin dosing algorithm 106 may be split between personal computing device 150 and drug delivery device 105 in any convenient manner and separate portions of insulin dosing algorithm 106 executing on personal computing device 150 and on drug delivery device 105 may communicate via wireless link 140.

Insulin dosing algorithm 106 may further communicate with cloud-based services 160 via communication link 142. In alternative embodiments of the invention, the insulin dosing algorithm 106 may be implemented in a cloud-based service 160 and accessed via wireless communication link 142 with the user's personal computing device 150. Further, cloud-based services 160 may provide a data store for storage of the user's past insulin use, for use by the insulin dosing algorithm 106.

As previously discussed, insulin dosing algorithm 106 may control the delivery of basal doses of insulin to the user at periodic intervals based on the specified basal rate. Insulin dosing algorithm 106 may comprise an algorithm which periodically cycles. For example, a cycle may comprise a 5-minute period during which a new blood glucose level reading may be received from sensor 180. The insulin dosing algorithm 106 may determine, in response to each additional blood glucose level reading if additional insulin is required above and beyond the insulin that is indicated by the basal rate and may control the delivery of the additional insulin during the current cycle. This additional insulin may be delivered as micro-boluses, which are insulin amounts above the baseline basal rate. Accordingly, the basal rate may not change, but additional insulin may be delivered as micro-boluses to address excursions in the user's blood glucose levels. Detection of excursions in the user's blood glucose levels may be used to adjust the user's TDI during the course of the pregnancy.

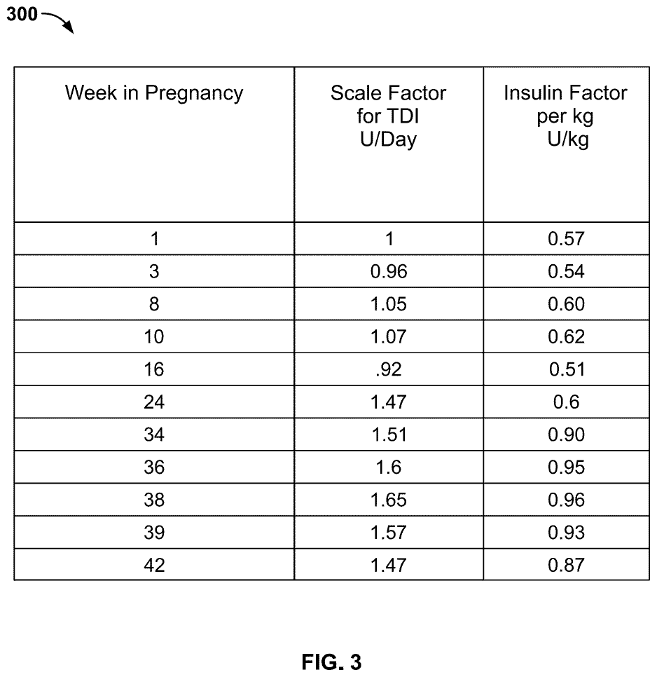

For pregnant women, as previously discussed, the TDI requirement will vary in various stages of the pregnancy. The user's pre-pregnancy TDI requirement (which varies on a person-by-person basis) may be used as a baseline which may be scaled depending upon the current stage of the pregnancy. FIG. 3 is a table showing scaling factors for various stages of pregnancy that may be used to scale the baseline pre-pregnancy TDI to provide an estimate of the appropriate TDI required for the current stage of the pregnancy. For example, in week 34 it can be expected that the TDI requirement is 51% higher than the baseline pre-pregnancy TDI requirement. The scaling factors shown in the table in FIG. 3 are average numbers over a large population of pregnant women suffering from Type I diabetes mellitus.

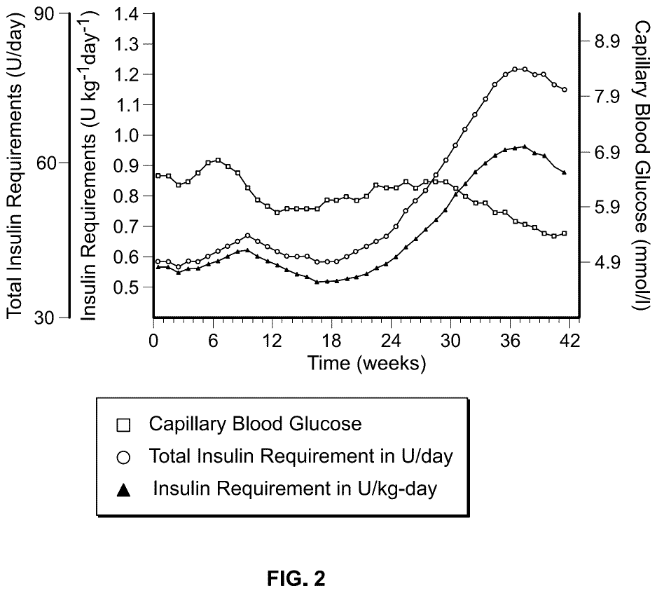

As can be seen from the graph in FIG. 2 and the table in FIG. 3, pregnant women are likely to experience a general rise in the TDI requirement from the baseline TDI requirement in weeks 1 through 8, followed by a decline from week 8 through 16 and then a continuous increase to week 36, followed by a decline up to week 39. The table in FIG. 3 shows the TDI requirement in U/day with a baseline of 1 and a notational scale factor for each week to baseline, as well as the U/kg insulin requirement.

In accordance with the first aspect of the invention, the insulin dosing algorithm 106 may periodically scale the user's TDI, for example, on a week-by-week basis, in accordance with the scaling factors in the table in FIG. 3 for each week (the “scaled TDI”). Other time periods for scaling may also be used and the scaling factors adapted accordingly. For the weeks of the pregnancy in between the weeks expressed in the table, a linear interpolation of the scaling factor may be used between weeks. In certain embodiments of the invention, the scaled TDI for any particular week may be considered an estimate and the user's blood glucose levels may be tracked to determine if the estimate provides satisfactory glycemic control. If not, the TDI can be adapted (the “adapted TDI”) during subsequent weeks.

For example, if the scaled TDI is set lower than the total insulin actually needed to provide satisfactory glycemic control, the insulin dosing algorithm 106 will deliver extra insulin by providing extra insulin as micro-bolus doses for the current time period and by increasing the TDI requirement to an adapted TDI requirement for subsequent time periods. Consequently, the adapted TDI for subsequent weeks may be adapted from the scaled TDI to a higher value than the value expressed in the table. For example, if insulin dosing algorithm 106 determines that 10% more insulin was required, the adapted TDI for the next week may be set 10% higher. Conversely, the opposite will occur should the insulin dosing algorithm 106 determine that the scaled TDI value is set to high. The determination of whether the scaled TDI provides satisfactory glycemic control may be determined by a percentage of time the blood glucose readings indicated blood glucose levels in a desired range. Thus, a threshold percent of the time that the blood glucose readings indicated blood glucose levels in the desired range may be used to determine the effectiveness of the scaled TDI. In various embodiments, the scaled TDI may be adjusted to obtain the adapted TDI based on various intervals of time, for example, adjustments could be made daily or weekly.

In accordance with the first aspect of the invention, drug delivery device 105 may be controlled by the insulin dosing algorithm 106 to provide basal doses of insulin such that the basal doses comprise approximately 50% of the scaled TDI or the adapted TDI requirement. The remainder of the scaled TDI or adapted TDI requirement may be used to administer bolus doses, either automatically by ADD 100 or manually by the user.

In a second aspect of the invention, insulin dosing algorithm 106 may be configured to detect when the user has ingested a meal, such as to be able to administer a bolus dose of insulin. In one embodiment, the insulin dosing algorithm 106 uses a machine learning approach for meal detection. The machine learning approach uses the blood glucose readings from sensor 180, which may be a CGM. In one embodiment in the training phase, the CGM data is labeled as being indicative of the ingestion of a meal when a rising blood glucose curve satisfies a rise of a first predetermined number of units in 20 minutes, a second predetermined number of units in 30 minutes, and a third predetermined number of units in one hour. For example, in one exemplary embodiment, the first second and third predetermined number of units may be 20 mg/dl, 40 mg/dl and 60 mg/dl respectively. In a second exemplary embodiment, the first second and third predetermined number of units may be 10 mg/dl, 20 mg/dl and 40 mg/dl respectively.

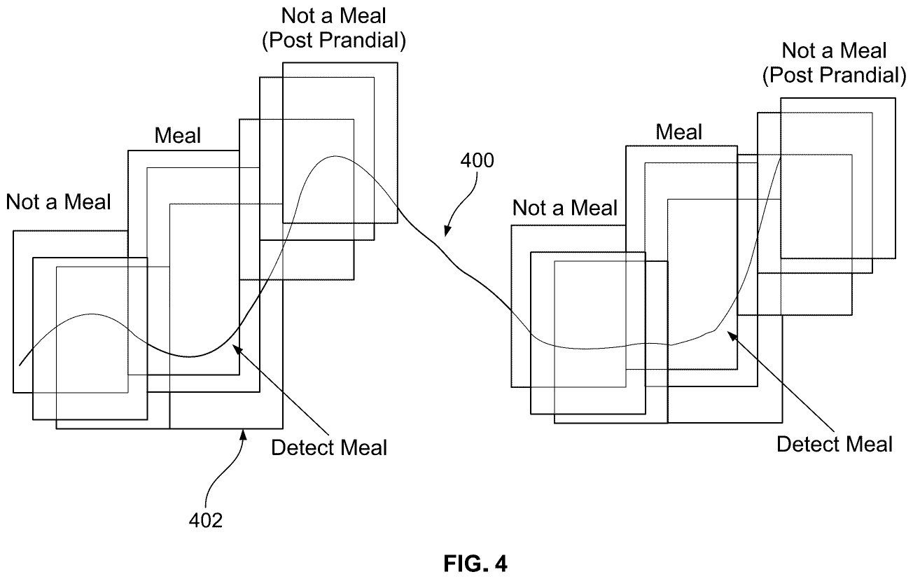

FIG. 4 shows the use of a sliding window 402 for extracting features from the blood glucose curve 400 for use by the machine learning model to make a determination of the ingestion of a meal. The features may be computed based on a sliding two-hour window that increments at five-minute intervals or whenever a new blood glucose reading is received. The features extracted from the CGM data (24 data points if a new blood glucose reading is received every 5 minutes) may include, in various embodiments, the first and second derivatives of the blood glucose curve at five-minute intervals (providing 48 features), averages of the blood glucose levels at 15-minute intervals (providing 8 features), the range of the blood glucose curve at 15 minute intervals (providing 8 features) and the slope of the blood glucose curve on 15 minute average blocks (providing 7 features), for possible total of 71 features. In other embodiments, the features extracted, as well as a number of features may vary.

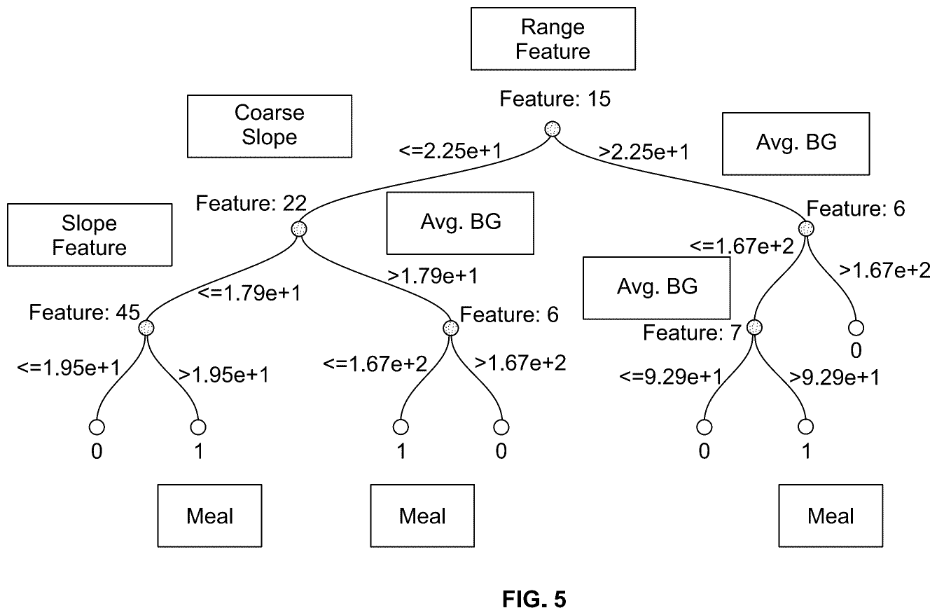

In some embodiment, the machine learning model may be implemented as one or more decision trees. FIG. 5 is a graphical depiction of an exemplary decision tree for determining meal ingestion, using the features extracted from the blood glucose curve as previously explained. Each decision point in the decision tree may be evaluated based on the value of an extracted feature. The decision tree may be set with values for the extracted features to delay the detection of a meal. For example, the decision tree may implement a meal detection model with the 10-minute delay that looks for a meal that started showing a rise in blood glucose levels 10 minutes in the past. Likewise, meal detection models having a 15-minute delay and a 20 minute delay look for a meal that started showing a rising blood glucose levels 15 and 20 minutes in the past, respectively. The results of multiple models may be cascaded or otherwise aggregated to increase the confidence in the determination of the ingestion of a meal.

In various embodiments, the meal detection may be implemented as one or more decision trees, as shown in FIG. 5. In other embodiments, a trained machine learning model, for example, a convolutional neural network, may be used that takes as input the user's blood glucose readings, or the extracted features from the blood glucose curve, and outputs a meal or no-meal decision.

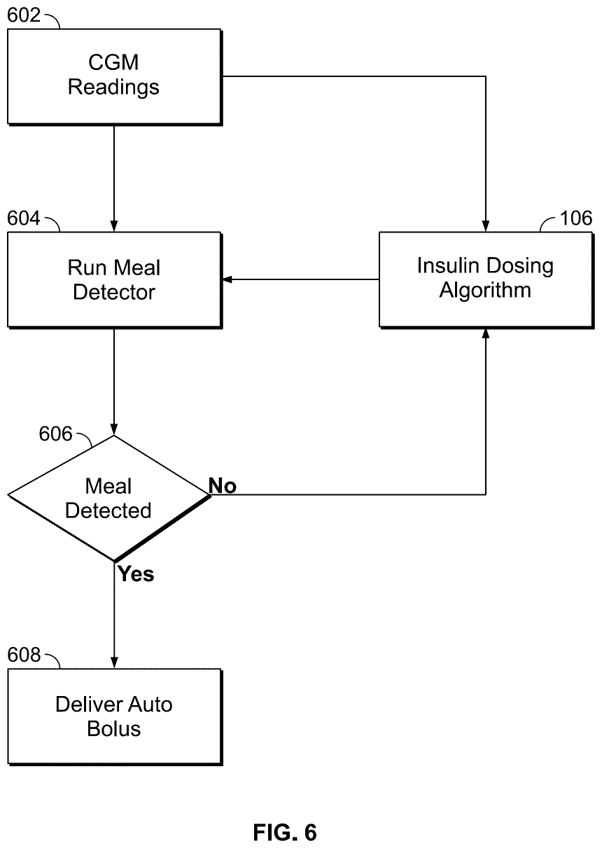

FIG. 6 is a flowchart showing the process by which meals may be detected. At 602, a reading is received from a CGM sensor 108. New CGM readings, in some embodiments, may be periodically received every five minutes, but other intervals are possible in other embodiments. Insulin dosing algorithm 106 is triggered by the receipt of the CGM reading to run the previously-described process 604 for determining if a meal has been ingested by the user. At 606, if process 604 determines that no meal has been detected, control returns to the insulin dosing algorithm 106, which waits for the receipt of the next blood glucose reading from the CGM. At 606, if process 604 determines that a meal has been ingested, in one embodiment, a bolus dose of insulin is automatically delivered to the user using drug delivery device 105.

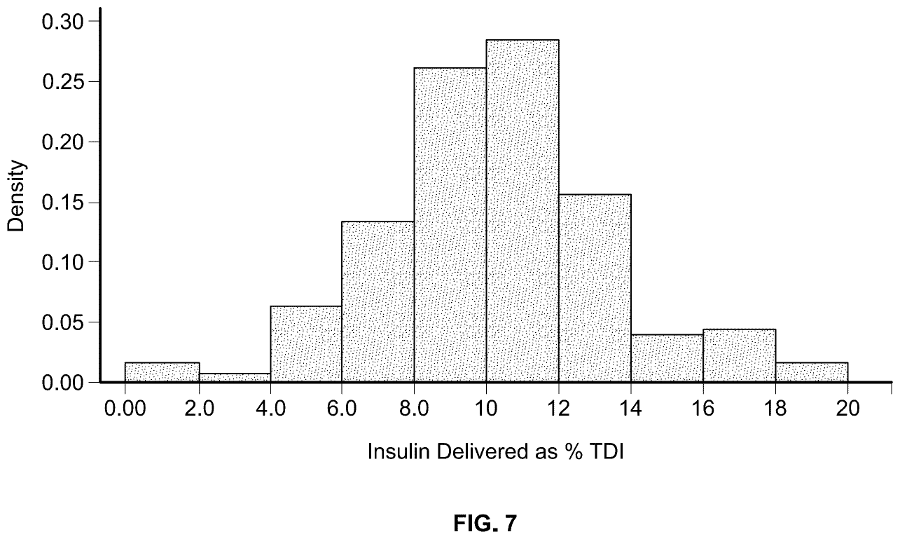

FIG. 7 is a histogram showing the size of the bolus dose as a percentage of the TDI, taken from a sampling of a population suffering from Type I diabetes mellitus. As can be seen from the histogram, most bolus doses are in the range of 8.0% to 12.0% of the TDI. It is therefore assumed that the range between 8% and 12% represents a typical bolus dose. A “safe” bolus dosage is one that is likely to avoid hypoglycemia, as it is a lower dose of insulin that will be delivered when the meal is detected per the flowchart of FIG. 6. Therefore, in some embodiments of the invention, the bolus dose administered by insulin dosing algorithm 106 is 8% of the TDI requirement. In other embodiments, other percentages of the TDI may be used to determine the size of the bolus dose.

The automatic detection of the ingestion of a meal, as well as the blood glucose readings received from CGM 108 are particularly useful in pregnancy because it reduces the user burden by not requiring the user to enter the macronutrient profile of the meal into a bolus calculator and it ensures that the user has eaten a meal and that the food is actually being absorbed before the bolus dose is administered. This is particularly important in the context of morning sickness or nausea, typically occurring in the first trimester, in which food which has been ingested may not necessarily be absorbed. This feature represents an improvement over prior art devices in which the ingestion of meals indicated by the user pressing a button on drug delivery device 105, which may cause a bolus dose of insulin to be administered without an indication that the food has actually been absorbed.

In various embodiments of the invention, various schemes may be used to administer the bolus dose. For example, in one embodiment of the invention, the automatic bolus dose may be administered as a single dose (e.g., 8% of the adapted TDI) delivered at one time. In other embodiments, the automatic bolus dose may be delivered in two phases, for example, an initial dose of 4% of the adapted TDI followed by a second dose of 4% of the adapted TDI if the meal is still detected after 30 minutes. In yet another embodiment, the automatic bolus dose could be delivered in multiple phases, for example, 2% of the adapted TDI as soon as the meal is detected, with an additional 2% delivered if a meal is still detected within one or more 20 minute intervals after the initial detection of the meal (not to exceed, for example, 8% of the adapted TDI in total). In some embodiments, the scheme used for delivery of the automatic bolus dose may be varied depending on the current trimester of the pregnancy.

In yet another embodiment of the invention, if the user has administered a manual bolus dose, the automatic delivery of the bolus doses may be disabled. Alternatively, a calculation of the insulin on board may be used to determine if the automatic bolus dose to be administered, or if the size of the automatic bolus dose should be modified based on the current insulin on board. The calculation of the insulin on board may take into account any manual bolus doses administered by the user, recent automatic bolus doses administered by the drug delivery device 105 and any insulin remaining in the body based on the administration of basal or bolus doses of insulin.

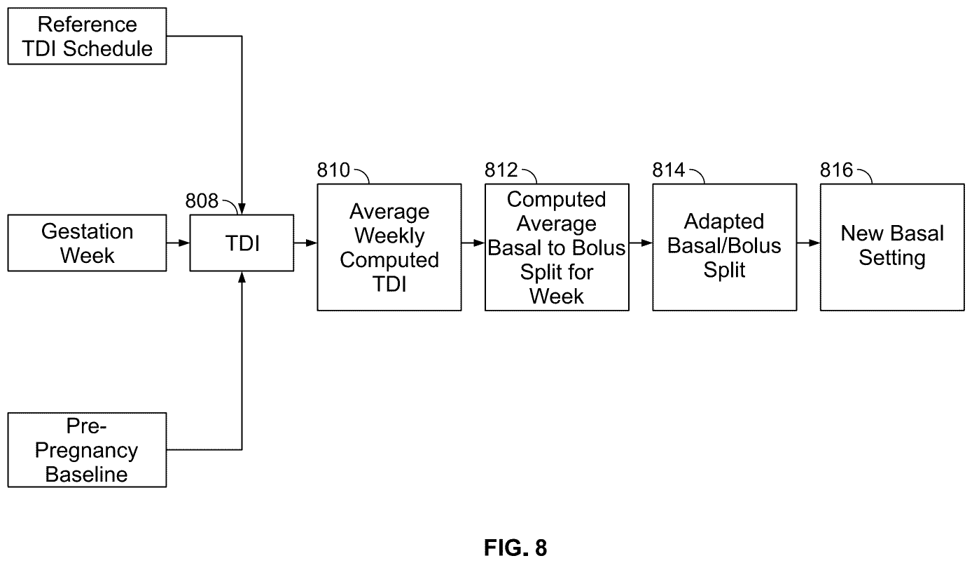

In yet another aspect of the invention, the split between the basal doses and bolus doses administered by the ADD system 100 as a percentage of the TDI, as well as a new basal setting (i.e., the percentage of the scaled or adapted TDI that is to be administered as one or more basal doses), may be dynamically adapted. FIG. 8 is a flow diagram indicating the various steps in the calculation of the new basal setting. First, the scaled TDI 808 based on the reference schedule 300 shown in FIG. 3 is calculated. The inputs required to calculate the scaled TDI 808 include the reference schedule 300, the gestation week and the pre-pregnancy baseline TDI. At 810, the average weekly scaled TDI 808 for the current week will be compared to the actual TDI delivery for the current week. The TDI for the next week (the “adapted TDI”) may then be suitably adapted based on the departure of the TDI from the reference schedule 300. For example, if the actual TDI for the current week is less than the scaled TDI 808 by 10%, the adapted TDI for the next week may be 10% less than the quantity indicated as the scaled TDI 808 or a fractional percentage of the deviation, such as reducing the adapted TDI for the next week by half of the deviation for the current week.

Referring back to FIG. 8, the pre-pregnancy basal to bolus split will be compared to the average basal to bolus split 812 on a week-by-week basis to determine if there are changes. The average basal to bolus ratio split for the week 812 is calculated based on the average adapted TDI 810. For example, if the pre-pregnancy basal to bolus ratio is 50%, this means that 50% of the TDI is delivered as bolus doses and 50% is delivered as basal doses. The insulin dosing algorithm 106 modulates the basal delivery based on the bolus doses administered (which manifests as insulin on board) as well as the blood glucose readings and the trend of the blood glucose curve. In the pregnancy mode, if it is found that the basal to bolus split is shifting (for example, to 60%), the basal to bolus split for the next week will be set to the adapted basal to bolus split 814, and the new basal setting 816 can be derived from the adapted basal to bolus split 814 as a percentage of the average weekly computed TDI 810. As an example, if the total of the bolus doses is equal to 40% of the adapted TDI, the remaining portion of the adapted TDI must be delivered as one or more basal does, such that the user receives the entire quantity of insulin indicated by the adapted TDI. Thus, the new basal setting for the subsequent week may be set to 60% of the adapted TDI.

In a final aspect of the invention, user feedback may be provided. In particular, differences between the scaled and adapted TDI through the weeks of pregnancy may provide several insights. For example, if the adapted TDI is higher than the scaled TDI calculated from reference schedule 300, this may indicate that the user is gaining a higher than average weight or that there is an increased insulin resistance. This may be communicated to the user, with suggestions for better control of food intake or increasing the amount of exercise. Conversely, if the adapted TDI is below the scaled TDI from reference schedule 300, this may indicate that the user is eating less than what they should be or is more active than average. If the user keeps a record of the meals that they are consuming by using a personalized meal interface, a catalog of all meals, the calorie content of the meals and the macronutrient profiles of the meals may be presented to the user as a review. If the user is wearing an insulin delivery device equipped with sensors to capture exercise information, a catalog of exercise activity may also be provided. Alternatively, the user may share their exercise information via a third party application. Other sensor-based metrics may also be reported, for example, sleep quality index. Weekly feedback and reports may be generated and provided to the user.

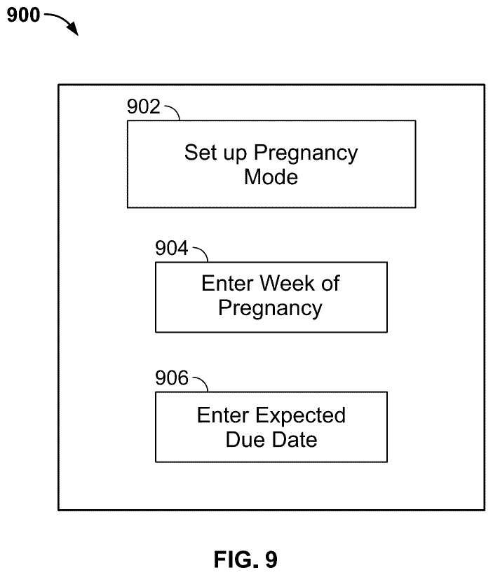

In one embodiment of the invention, the process of scaling and adapting the TDI during pregnancy, as discussed above, may be implemented as a “pregnancy mode” of insulin dosing algorithm 106. The software of a typical prior art ADD system 100 can be modified to include the pregnancy mode and any or all aspects of the invention discussed above. In an alternative embodiment of the invention, a special version of ADD system 100 may be configured for use during pregnancy. FIG. 9 shows an exemplary screen 900 from a user interface which allows the user to invoke the pregnancy mode. Pushing button 902 may initiate pregnancy mode. Because the calculation of the scaled or adapted TDI and the basal to bolus split is based on the gestation week, the user may enter either or both of the week of pregnancy, by pushing button 904, or the expected due date, by pushing button 906. Screen 900 may be displayed in user interface 158 on personal computing device 150, as shown in FIG. 1, and communicated to drug delivery device 105 via wireless communication link 140 for use by insulin dosing algorithm 106. Insulin dosing algorithm 106 may have knowledge of the user's pre-pregnancy TDI requirements, as they may be stored in a data store on cloud-based services 160, or may accept the user's pre-pregnancy TDI via user interface 158. In certain embodiments of the invention, pregnancy mode may end upon the onset of labor or when delivery of the baby is completed.

The foregoing has been presented for the purposes of illustration and description. It is not intended to be exhaustive or to limit the present disclosure to the exemplary embodiments disclosed herein. Many modifications and variations are possible in light of this disclosure. It is intended that the scope of the present disclosure be limited not by this detailed description, but rather by the claims appended hereto. Future filed applications claiming priority to this application may claim the disclosed subject matter in a different manner and may generally include any set of one or more limitations as variously disclosed or otherwise demonstrated herein.

What is claimed is:

1. A method comprising:
displaying, on a display of a computing device, an interface screen configured to interface a user with a drug delivery device, the interface screen comprising a selectable element configured to set the drug delivery device into a pregnancy mode upon selection;
receiving, at the computing device, a user input specifying one or more of the user's week of pregnancy or an expected due date;
accessing a pre-pregnancy total daily insulin requirement of the user;
scaling the pre-pregnancy total daily insulin requirement by a factor as a function of the week of pregnancy or the expected due date to obtain a scaled total daily insulin requirement; and
controlling the drug delivery device to deliver one or more doses of insulin based on the scaled total daily insulin requirement.

2. The method of claim 1, wherein the pre-pregnancy total daily insulin is received at the computing device from a user input.

3. The method of claim 1, wherein the pre-pregnancy total daily insulin is retrieved from a cloud storage device.

4. The method of claim 1, further comprising:
obtaining a plurality of blood glucose readings from the user;
determining that the scaled total daily insulin requirement does not effectively control the blood glucose level of the user, based on a predetermined percent of the blood glucose readings of the user being outside of a desirable range; and
determining an adapted total daily insulin requirement by adjusting the scaled total daily insulin requirement up or down based on the determination that the scaled total daily insulin requirement does not effectively control the blood glucose level of the user.

5. The method of claim 4, further comprising:
determining, for a predetermined period of time, a percentage of the scaled total daily insulin requirement above or below a level necessary to effectively control the blood glucose level of the user; and
adjusting the scaled total daily insulin requirement for a next predetermined period by the percentage or a portion of the percentage to obtain the adapted total daily insulin requirement.

6. The method of claim 4, wherein a first predetermined percentage of the adapted total daily insulin requirement of the user is delivered as one or more basal doses.

7. The method of claim 6 further comprising:
tracking, for the predetermined period, a split of the adapted total daily insulin requirement for the predetermined period between basal doses and bolus doses; and
adjusting the first predetermined percentage of the adapted total daily insulin requirement of the user that is delivered as basal doses during a next predetermined period such that the total insulin administered to the user during the course of a day is the adapted total daily insulin requirement.

8. The method of claim 6, further comprising:
automatically detecting an ingestion of a meal by the user; and
administering a bolus dose of insulin in response to the automatic detection, wherein the bolus dose is a second predetermined percentage of the adapted total daily insulin requirement of the user.

9. The method of claim 8, wherein the bolus dose is delivered as multiple doses wherein a first portion of the bolus dose is delivered upon detection of the ingestion of the meal and a subsequent portions are delivered if an ingestion of a meal is detected after multiple predetermined periods of time.

10. The method of claim 9 wherein a total of the bolus portions of the bolus dose administered do not exceed the second predetermined percentage.

11. The method of claim 8 wherein the automatic detection of the ingestion of a meal further comprises:
receiving, from a sensor, periodic readings of the blood glucose levels of the user;
constructing a blood glucose curve for a moving window comprising a predetermined number of the blood glucose readings;
extracting, from the blood glucose curve, one or more features; and
determining, based on the extracted features, that a meal has been ingested by the user.

12. The method of claim 11, wherein the extracted features are used in one or more decision trees.

13. The method of claim 12 wherein the one or more decision trees have varying delays built in such that meals are detected by each decision tree after the respective delay of the decision tree, the delay indicating a period of time after the blood glucose curve shows an initial rise in the blood glucose levels of the user.

14. The method of claim 13 wherein the two or more of the decision trees having different delays are aggregated to form a cascaded model.

15. The method of claim 11, wherein the extracted features are input to a machine learning algorithm and wherein the machine learning algorithm outputs a meal or no-meal decision.

16. The method of claim 11, wherein the extracted features include one or more of: one or more averages of portions of the blood glucose curve, one or more ranges of portions of the blood glucose curve, one or more coarse slopes of portions of the blood glucose curve, one or more slopes of portions of the blood glucose curves and one or more second derivatives of portions of the blood glucose curve.

17. The method of claim 1 further comprising:
providing feedback to the user when the adapted total daily insulin requirement deviates from the scaled total daily insulin requirement.

18. The method of claim 17 wherein the feedback comprises information regarding food intake and exercise.

19. The method of claim 1, further comprising:
accessing a catalog of meals ingested by the user, the catalog including quantities and macronutrient profile of the meals; and
recommending modifications to meals ingested by the user based on the deviance of the adapted total daily insulin requirement from the total daily insulin requirement.

20. A non-transitory computer-readable medium storing instructions that, when executed by one or more processors, cause the one or more processors to:
display, on a display of a computing device, an interface screen configured to interface a user with a drug delivery device, the interface screen comprising a selectable element configured to set the drug delivery device into a pregnancy mode upon selection;
receive, at the computing device, a user input specifying one or more of the user's week of pregnancy or an expected due date;
access a pre-pregnancy total daily insulin requirement of the user;
scale the pre-pregnancy total daily insulin requirement by a factor as a function of the week of pregnancy or the expected due date to obtain a scaled total daily insulin requirement; and
control the drug delivery device to deliver one or more doses of insulin based on the scaled total daily insulin requirement.
